SEQUENCE LISTING (20 sequences)

SEQ ID NO: 1 - DNA, 19 bp, Zea mays
tccctatcagtgatagaga

SEQ ID NO: 2 - DNA, 17 bp, Zea mays
tttcgttggaaacggga

SEQ ID NO: 3 - DNA, 21 bp, Zea mays
aattgtgagcggctcacaatt

SEQ ID NO: 4 - DNA, 20 bp, Zea mays
tactgtatatatatacagta

SEQ ID NO: 5 - DNA, 17 bp, Zea mays
cggaggactgtcctccg

SEQ ID NO: 6 - DNA, 157 bp, Zea mays
gagtaccgctagtccctatcagtgatagagatgcagtcgccgaatactgtatatatatacagtaagccttctactggaattgtgagcggctcacaattagtaggatgaaattttcgttggaaacgggacatatgccttctcggaggactgtcctccg

SEQ ID NO: 7 - DNA, 1083 bp, Escherichia coli
gtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtga

SEQ ID NO: 8 - DNA, 1145 bp, Escherichia coli
  Amino acids 1-72
tgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatccggtcgcgcgaattgttaattaagcgatcgctatttaaataggcgcgccagtttaaacaggccggcctaaggtacccggggatcctctagagtcgacctgcaggcatgcaagcttgggatctttgtgaaggaaccttacttctgtggtgtgacataattggacaaactacctacagagatttaaagctctaaggtaaatataaaatttttaagtgtataatgtgttaaactactgattctaattgtttgtgtattttagattcacagtcccaaggctcatttcaggcccctcagtcctcacagtctgttcatgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatcgcggccgcctagagggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaaga

SEQ ID NO: 9 - DNA, 2646 bp, Saccharomyces cerevisiae
  Amino acids 1-74
ttactctttttttgggtttggtggggtatcttcatcatcgaatagatagttatatacatcatccattgtagtggtattaaacatccctgtagtgattccaaacgcgttatacgcagtttggtccgtccaaccaggtgacagtggttttgaattattaccatcatcaattttactagccgtgatttcattattcatgaagttatcatgaacgttagaggaggcaattggttgtgaaagcgcttgagaatttgtttgagttgttatgaggttcggaccgttgctactgttagtgaaagtgaaggacaatgagctatcagcaatattcccactttgattaaaattggcgccaccaaacaaagcagacggggtcagtggcactaatgattgcagctgttgctgttgccctagaaaaggcgtgactgagcgatgcgaaggtgtgcttcttggtattgtcactggagagttacgagagggtggacggttagataacagcttgactagatcactgaaacttgctcctgatttcaatggcacaggtgaaggccctactgagccaggagaaacatatttaacactgatattgttgacattttcctccggaagagtagggtattgggcgatagttgcagaaccgacaatatttttaatggcgctaccattactattgttataactgatatgcggtaatgggattgcacactgtgataacagaaacggcgcacatacctcttccagtacttgaatgtatttttcacaagtctggattttaaaagtggccagtttttttaatagcatcagaacagtgttaatttgttgtaataattgtgcggtctcgttattctcagcattcgattttgagtttgagagtagagtctttatgggtactaggactgcattgaacaagtaataagaacaattccaggcaaaatatggggtgacattatgattgtccatatagctacttacagacataacagttctttgtgctgcatcgcttaacatgatggagcatcgtttaacttcataactttgatgatcattttgatcctgttctagttgtgactttttctgggtaaaattagtgaaaaaatctcttaatacataaatgataagagacaactgtttccacttcagttcgaatcttgtaaaggatagccaagggtgttccttcaacaaattggttagagcggtggtggaaatatccatttgtaaaaactttggtgcctgtctcgaaacctcctcaatctcattacaaatcatcaagcatttttttgcacatataggacttttttctgcagttactgttttgtctagttcatagatttttgtgaaaacttgtaagagccttgctgtttcaatgatgccatgatatatggtgggacctgttgtggtacgctgcacatcgtcgacagaagaagggaaggagattgtattctgagaaagctggatggatcgaccataaagcagggacaattggatctcccaagagtagacagaccaccaaattcggcgtctttgttccagaatgctgctatcactgaaggacgaggggaggtccctattcaagcccaatgatatggccattcttatggaaaagctgtgaaaattatagctagtatttgttttctgcctccactgtgtatatcgcgacagaagatgtagggctgtcaccaaaattatggaacctgactcgaagaccttgctcgtcaaatgagatttagcattttgatagtaaaaaacatctatatcagtagattccccctctatacaccaggctccaatggctaatatgcagttaaaaaggatttgccattgatccttcgacgcgatttcaatctggttattatacaacatcattagcgtcggtgagtgcacgatagggcagtaggggtgaaaattattgagataactttgaagtaaacgggatgttgtggatctagaagccaacgtgtatctatccgtaatcatggtcgggagcctgttaacgttagagttcgtgtaattttccggtttaaagccaatagatcgaagaatacataagagagaaccgtcgccaaagaacccattattgttggggtccgttttcaggaagggcaagccatccgacatgtcatcctcttcagaccaatcaaatccatgaagagcatccctgggcataaaatccaacggaattgtggagttatcatgatgagctgccgagtcaatcgatacagtcaactgtctttgacctttgttactactctcttccgatgatgatgtcgcacttattctatgctgtctcaatgttagaggcatatcagtctccactgaagccaatctatctgtgacggcatctttattcacattatcttgtacaaataatcctgttaacaatgcttttatatcctgtaaagaatccattttcaaaatcatgtcaaggtcttctcgaggaaaaatcagtagaaatagctgttccagtctttctagccttgattccacttctgtcagatgtgccctagtcagcggagaccttttggttttgggagagtagcgacactcccagttgttcttcagacacttggcgcacttcggtttttctttggagcacttgagctttttaagtcggcaaatatcgcatgcttgttcgatagaagacagtagcttcat

SEQ ID NO: 10 - DNA, 636 bp, Escherichia coli
  Amino acids 1-206
atggcacgactaagcttggacgacgtaatttcaatggcgctcaccctgctggacagcgaagggctagagggcttgactacgcgtaagctggcgcagtccctaaaaattgagcaaccgactctgtattggcacgtgcgcaacaagcagactcttatgaacatgctttcagaggcaatactggcgaagcatcacacccgttcagcaccgttaccgactgagagttggcagcagtttctccaggaaaatgctctgagtttccgtaaagcattactggtccatcgtgatggagcccgattgcatatagggacctctcctacgcccccccagtttgaacaagcagaggcgcaactacgctgtctatgcgatgcagggttttcggtcgaggaggctcttttcattctgcaatctatcagccattttacgttgggtgcagtattagaggagcaagcaacaaaccagatagaaaataatcatgtgatagacgctgcaccaccattattacaagaggcatttaatattcaggcgagaacctctgctgaaatggccttccatttcgggctgaaatcattaatatttggattttctgcacagttagatgaaaaaaagcatacacccattgaggatggtaataaatga

SEQ ID NO: 11 - DNA, 1800 bp, Homo sapiens
  Amino acids 1-125
atgggccccaagaggcgacagctgacgttccgggagaagtcacggatcatccaggaggtggaggagaatccggacctgcgcaagggcgagatcgcgcggcgcttcaacatcccgccgtccacgctgagcacgatcctgaagaacaagcgcgccatcctggcgtcggagcgcaagtacggggtggcctccacctgccgcaagaccaacaagctgtctccctacgacaagctcgagggcttgctcatcgcctggttccagcagatccgcgccgccggcctgccggtcaagggcatcatcctcaaggagaaggcgctgcgcatagccgaggagctgggcatggacgacttcaccgcctccaacggctggctggaccgcttccgccggcgccacggcgtggtgtcctgcagcggcgtggcccgcgcccgcgcgcgaaacgctgccccccgcaccccggcggcgcctgccagtccggccgcggtgccctcggagggcagtggcgggagcactactggttggcgcgctcgggaggagcagccgccgtcggtggccgagggctacgcctcgcaggacgtgttcagcgccaccgagaccagtctatggtacgacttcctgcccgaccaggccgcggggctgtgcggaggcgacggacggccgcgtcaagccacccagcgcctgagcgtcctgctatgcgccaatgccgacggcagcgagaagctgcccccgctggtggccggcaagtcggccaagccccgcgcaggccaagccggcctgccctgcgactacaccgccaactccaagggtggtgtcaccacccaggccctggccaagtacttgaaggccttggacacccgaatggctgcagagtctcgccgggtcctgctgttggccggccgcttggctgcccagtccttggacacctcgggcctgcggcatgtgcagctggccttcttccctcccggcaccgtgcatccgctggagaggggagtggtccagcaggtgaagggccactaccgccaggccatgctgctcaaggccatggccgcgctagagggccaggatccctcaggcctgcagctgggtctcacggaggccctgcactttgtggctgccgcctggcaggcagtggagccttcggacatagccgcctgctttcgtgaggctggctttgggggtggccctaatgccaccatcaccacttccctcaagagtgagggagaggaagaggaggaggaggaggaagaagaggaggaggaagagggtgaaggagaggaagaggaggaggaaggggaggaggaggaggaggaagggggggaaggagaggaattgggggaggaagaggaggtggaggaggagggtgatgttgatagtgatgaagaagaggaggaagatgaggagagctcctcggagggcttggaggctgaggactgggcccagggagtagtggaggccggtggcagcttcggggcttatggtgcccaggaggaagcccagtgccctactctgcatttcctggaaggtggggaggactctgattcagacagtgaggaagaggacgatgaggaagaggatgatgaagatgaagacgacgatgatgatgaggaggatggtgatgaggtgcctgtacccagctttggggaggccatggcttactttgccatggtcaagaggtacctgacctccttccccattgatgaccgcgtgcagagccacatcctccacttggaacacgatctggttcatgtgaccaggaagaaccacgccaggcaggcgggagttcgaggtcttggacatcaaagctga

SEQ ID NO: 12 - DNA, 474 bp, Zea mays
atggctcgaaccaagcaccaggccgtgaggaagacggcggagaagcccaagaagaagctccagttcgagcgctcaggtggtgcgagtacctcggcgacgccggaaagggctgctgggaccgggggaagagcggcgtctggaggtgactcagttaagaagacgaaaccacgccaccgctggcggccagggactgtagcgctgcgggagatcaggaagtaccagaagtccactgaaccgctcatcccctttgcgcctttcgtccgtgtggtgagggagttaaccaatttcgtaacaaacgggaaagtagagcgctataccgcagaagccctccttgcgctgcaagaggcagcagaattccacttgatagaactgtttgaaatggcgaatctgtgtgccatccatgccaagcgtgtcacaatcatgcaaaaggacatacaacttgcaaggcgtatcggaggaaggcgttgggcatga

SEQ ID NO: 13 - DNA, 2106 bp, Zea mays
atggacgccgccgaccccctctgcgccatctcctcgactgcgcgactccttccgcggacccttggccccgccattgggccttccccctcgaacccccgcgacgcgctcctcgaggccatcgcactcgcccggtctctgaaagggtctgaggagctggttaagcaggctacaatggtgccgaaggagcacggggacatccaggctctctaccatgatgatggagtgaaaggctggcctcctgcaaatggcagcaaagagcaacagggaagaaggccagcactggatcgtaaacgggctcggtttgctatgaaggacactggaagcaaaccggtgcctgttgtggaccaatctaagttatcaaatatttcagatccaatcacattcttcatgaccttggatcggcttgaagaagccgaggaggagatcaaacggctaaatggagaggcagaaaaacgcacattgaattttgatcctgtagatgaacccattagacagcctggtttacgtgggagaaaatcagtccgcagtttcaaggtaattgaggatgttggtactcaagatcccaatgaggcaccggcttcccaaacagcaactatgacaggatctcagttgtcacaggatgttatgcatgctgttgcgggcaaaaatggacgatctgtttcctcaagatctagtgaagctatttcagagaaagaagtttcattagctgagaaggatggccgtgacgatttgacttatatactgacctcaatccaagatttagatgaatctgaagaggaagagtttatccggaagaccttagggattaaagaaacaaggaaagaaagagttagtcttcgtaactccattcttggagttaggtccctaagaagcaatactgaacaaaaaggttcaatgaaggtccaccctccagaaagtcatttgcctcagactcgccaagatcgaatttcagagttagagaaacatttgtttcctggaggtgcagcaaatgctaaatgcacagatgatgaatctgaagggtcaccagatattgtgatgggtgaaccatcattggtgcattattcttctgatgttctgatgactgatgagaactttactgcaagtgacgttgacagggagaccccaaatctgggtgccagagcagcagaccctgttcttgatcctgaaccaaacttgcctgatcatgcatatgaaaggcaacctggaggttcctcacttggtttgtacagagacacagaagttggagttgccaaagaaaatgagacatgtaacaggagcaatatatctgtggaggaagatgatgtgccaatagactatataactattggcaggtctactaatgaaacagaagtttcttcttctcatcctttggagggcagctcgacagaagaattggtcactaaaccaggtagacatgcggctccagatggtatctgtaggacttcacatgcagctgaggatagcattcaacatctagaagcggtcaaagagggtggtgtcctacaagataagtcaagccagttgttggagatgcctcaggaagatattaatccactgaatcaggctcagatgcatggtggaagcactaagaaatcggcacctgatctatccccgaccaaacagaagaagcaacaggcagttcaagaaaggaaaaggaagcaacagtcaaagaggggcaagaaagtggctggtgaatccctggaaataccccaaacaaattttgaatcggagaatcaacctcataatgacgatgtcaacattgagaaacagacaattacgagtagcacactctcaccaaatggtgccaaggggcaaaagggagctcaaaggagaaaccagaccaaaaaaaggaatcaaagaaaaatccttggagatgctgaccttgcatgccagcctggagtgagaaaaagctcaagaacacgctcaaggcctttggagtattggcttggtgaaagattactatatgggcctatacatgacaatttgcatggagctattggcatcaaagcatactctcctggccaagatggcaagagatcattgaaagtgaaatctttcgtgcctgaacagtattcagatcttgttgctaaatctgcaaggtactga

SEQ ID NO: 14 - DNA, 883 bp, Zea mays
acctctgaagagtcggaagaagaagcgggcgatggacgacggcgacgagagcgcggccgcggcggcggcgctggggctgagcccgcagcttttcgtcaacgaggtccacggcatcatagccgacatcagcgccgaggccttcgagtactgtctccaggcagcagccgcgccaggtgtcgtcggcgccgccacggctgcagagaaggccacggatctccagcggggattaaatgctattcaccatgttgtaaaggatcgactggataagagaatgaccaactgggagaaattctgctatcgacattgcttcgatgtacctgaaggttttgtggccgctgatgatgatagttcttgtgcaaatgaatcacaaaaagatgggatttcggattcagatttggatttggaacttgactctttgaggagaaagtttggaagtgctaacaaggaatctgaaaaccttgaaagagagttgtcctcactgggaaggcaagctacgttcaaaagaagacttgattcttctgtctctgagatagaaaagttgtttgaagaagaaatggtgcagggaaatttgaaagatcttgtgaaggcaataccggttttgcaccagaaaataatagatgcaaatcagagaaggactgagattaggagcttggtagatcaacaggcttggaacacgaagagatcgagagacagcaagccataagcgctggagaagacaggttctactgcatgtgttgaagacattcaagaggttgtcaacatcttacagagcaagtagcttgttgctcttttcctgattcatgttggttccacatagcgcctgcagcttgcttgcttggataaagcagtggtttgcccagttagtcttgttattgatcaaccg

SEQ ID NO: 15 - DNA, 1503 bp, Zea mays
ttcggcacgaggctttctcacccttcatccaacggcgccagcacgcacgagcagtgcgccgccatccgttgcgccgtcgtacgcagccagctggagcgccgcttcccgccagccagccggagcgccgccgcccgcgagagtcccgcccccggcgtgtatcagaaccccccccctcccgggcgccgcgcagcagagctccgtggcccccgccctcgccgagcagcagaggaccgcctccagccagctcgccggctatggctgccgctggagatccaccacctcctatttatgctgagggtgctgcaaggacaagaggagaaatatctccacatttccagacatatgcaagggacgacgatgcaaaacttgagaccacacgtgcaaggctgtcaaatatcctcaaaaggcatgaagatctgaaagagcgcctttcacgggattctgataagctcatttttgagcgcctacagaaagaatttgaggctgcacgggctgctcaaaccgaagagatatcaataaatgatgatgagtggaatgatggtttactggcaaccatccgtgagaaggtatttaatgttcatatggaggctgataggaaggccatgtccaaccaagtaaatgttccagcagatctttcatttcaatcgaggactacatataggattagaaataaggttatctattgcttggatggagctaggattggcatacagtatgagacattctttgctggagagccttgtgaaatttttcattgtgttttggaaagcaagtcattcctggaaaagatgattatgattgaacacaccttacctttctttctgcctaaattcattgatcatcttgaagaaattttacaggcttatattgacaggagagagcaggttcgtcttatcaaggagctctacgggaaccaaattggcgagctgttccatagtcttccttataatatgatagagtttgtattggaggattttgagtgcaaggttacaatcagtatccgatattcggatctactgcagaccctgccgagtcaggcaagggtcttggcttggccacttcgctcggccaagaaaatatctacaagaagcagcagtgcatcagctgcgcaaccagtaccctttcacctttcatatgccgaggaagcactcaaaaccttgtgtctaccagaagcatacgcagacattgtgctggagctaccccatgctttgaagaggattttatcctctcgggacagtgactaaatcgtgacctcaagatgtccagttttgttgtgcataagattcacatctcaatcgtggggcatgtttttgaagatattgttcagaatcttgtggtagtatagcctgcagttgtggactgcaattcctgtgaaagcaaggcagacatttgcgtgggccgctcgaaatattccggctacagttcctgttaaaagaatcaaaatcctaggaaggccttatggtcgaggcataatcttgccaaaaaaaaaaaaaaaaaaaaaaa

SEQ ID NO: 16 - DNA, 1731 bp, Zea mays
atgcggcgcggcggcggtcgccggttccccaagccctccttggcgccctccaccgcggccgaggccaccccggcgctggacgccagcgtcatccgcaacctcgactccgccttctcccgccgcgactccgacgccaacagcctctgcagcagccgtcccgcttctaccgtcggcgccatcctcaacttctccgaccgcgccacgcaggtcgccgcgctccgcgtcgtcaacacattcctcgcccccgccgtcacgctccgggggcctctgcccgccgcgcgcgacatccaggcggcgctccgcctcctcgtcgaccgtctccacttgccccgcaacgacgccacattcgatgatgacctcatccaggaccttcgccttctcgggtgcccctacaaggtcacccgctccgcactcaaggcccccggcaccccgcactcgtggccggtactgctctccgtactccactggctcaccctcctctgccactcccaaggagacgacccggatgccccgagcggcccatccgacgatctcttgctctacatcacccagtcgtactcccacttcttgtcgggggacgacgccgccgtcgaaaccgtcgacgaggagtacgcctccaaagcccggatgaccggtgaggcgtacgttgcaacggctcgtgccctggagaaggaggctgaggaactggagacccaggtgaataagctcatctcgggtccctcgcggcgggaagcactggagtcagagaaggaggccttcaatgctgatattcacaagttcgacgcagtggtgaatgcttggaaaactaagttcaatgaaagggaacaatcgttgggggatttggagaaagagttggaggcaaaagtgtcggacacccagcgcgctgctgctgaaattcaggatctattgaaacaagtggatgctcagccggtgaatgtgaaggatgtggatagaatgcgtagggagatgcaggctattgaagatgacattgccaacgccgagaaggggaaaacagcactggaggataaggtctgggaacttgaagctaagctggttaccaagcttgaggagcttgagaggcatgctgagcagtgcaaccaggccctcaaaaagttgaaacctactgttgcttttcagtacatgatagattcaaaaggatcttctcctgctgagatgctaggtactggttacaaaacagtactaaaacctgcacttctggctcatgctgaggagaacaaaaggatctgtctctcaaatcttgaaaacttaaatgatctacagaagcagctgcaaggaaatgctaaggttctagaggaggaaaggaataatatttccagtctccaggaaaaaaatgacgaaatcgttgctcgtttgaatttactggatcgtgaaattataaatgatgactcaagattcacatctgaagctgcacaaatgaaagatgagttggagaaaaagaagaatagtttgatttctgctgaaaagcaggcagatgattttcttaagaaatcggagaagaggctccaagatgtaacacttaaagttgaagaagaaactcaggcagctgctaaggagctgctggagcttcttgattccatggctgaacataaagagttcatggaaacaacaattgctcagaggaggaaggacctctatgaaactgcagattatatagcatctgtggcatccaagacagtcatcacccaagtaccgaaccctgctgaatcctga

SEQ ID NO: 17 - DNA, 1131 bp, Zea mays
caaaagcgtcgcttcgtgctagtatgcgaaaacttgtgttatttaactttaattggaaaaccatctctttgtccaacgccgactccctgagccactgacacggtgacgccacactcacgaacggcgaaaccccaccaggccaccacgcacaaccgctccaaccatggacgccggcgccgcgcgtccatcggcaccgtcgaccgccgcagtcgcgggggcctccgtcgcggacgagccccgggacgcgcgggtggtgcgggagctcctgcgctccatgggtctccgcgagggcgagtacgagccccgcgtcgtgcaccagttcctggacctggcctaccgatacgtcggagacgtgctcggggacgcccaggtctacgccgaccacgccgggaaggctcagatcgacgccgacgacgttcgcctcgccatccaggccaaggtcaatttctccttctcccagccgccgccgcgcgaggttctccttgagctggcccgtagccggaacagaatgccgctgcccaaatcaatcgctcctcctggctcgattcccctcccacctgagcaggacacactgttggcccagaactaccaactcctgcctccgttgaagccgccacctcaatatgaggaaatagaggatgagactgaagaaccaaacccaagtaacccggcgaattctaatccatcctattcgcaggatcagagtagtaaggagcagcaacagcagcacactcctcagcatggtcagagggtttccttccaacttaatgctgtggcagctgcagctgcagctgcaaagcgtcctcggatggccattgaccagctgaacatgggctaaccgtttcgtcggttagactgtagtatctttccccccaccctctagtctggtgaatcatgttttgaagagatagtgctgtagtggtatatgctcttactgactaccacccacatatttaaccacgagatgtgcagtccaacagcaccagcagcataagtttctgcctgcaagaacgttgctggagggaaggaaggaaagtgactgttctacagttctagtagtgtattttgtacttgcacactaaaaggttaatatctgctagtgtaattttaaaaataaaaaaaaaaaaaaaa

SEQ ID NO: 18 - DNA, 1106 bp, Zea mays
ctcttctccagcgtccgatctctcccactcgccttcctcaccgcagctctcccggctcggtcgcttcgccacctccgtcctccccccgcgctcggtcgctcgccacctgctctcccctccctccacgttgctcgcgcccgcgcttatataagtgcaggaggagctcatggcggacgctccggcgagccctgggggcggaggcgggagccacgagagcgggagccccaggggcggcggaggtggaggcggtggcagcgtcagggagcaggacaggttcctgcccatcgccaacatcagtcgcatcatgaagaaggccatcccggctaacgggaagatcgccaaggacgctaaggagaccgtgcaggagtgcgtctcggagttcatctccttcatcactagcgaggcgagtgacaagtgccagagggagaagcggaagaccatcaatggcgacgacctgctgtgggccatggccacgctggggtttgaggactatattgaacccctcaaggtgtacctgcagaagtacagagagatggagggtgatagtaagttaacttcaaaatccagcgatggctccattaaaaaggatgcccttggtcatgtgggagcaagtagctcagctgtacaagggatgggtcagcaaggaacatacaaccaaggaatgggttatatgcaacctcagtaccataacggagatatctcgaactaatgaagacatggaccttttctgcaacagctgctcttccccgaggcgggtttttggtcgcagttatttactaagtaagacaccttgcggtgaccattaaagagtaaccaatcaccctgggtaggtcaatttttatctgcaagaactgatgaggccgcttggtaggagtaaatcgcttttcctgggaacgattgttggttagcgccgctactgtatgtatattgagataccttaacgattggtcttttggctgccatttggttacatgtatttgtatttggaggcataagtatcgtgtaatttgtgttatgactagtgtattgactattgaattatgagaagagctgctttagttgtaagatcacatgacacatccaagaattcccaaaaaaatgttcgt

SEQ ID NO: 19 - DNA, 864 bp, Zea mays
cccggcggcggcggatctcggaggcagcgacgcgcgggtggaggagctcagtgacagcgccagtgcctatcgagcgagcgaatcgaggagattctccccactgtttctttcctgctgattccgatttcgcgttgaaacaccggggggagaagaattgcgggcgaccataactcggcaagaagcggctccaaaaagggcatctacctttgccgattccggcaccgatggatcccgcggcgtcggggaggttcgacaagctccagagctcgttcaagctctccatccagtgcctcctcaccgcctgctcccgcgaggatgtcaatgacgctttctcgtccttcactgatgctgagaaggagcgtttgcaccggatgctcacccttgtgatgaagaatctgcatgccaacgtagtggatgagtttgacgacttctgccaagaaacacaggtggctgctgcccttgaaaagatagatgactttgttgaaaagcagaatcttgatgcattatcttcagagaagactactgttgaagaaatcgaagaaaaggtatcaagagcaaaaaaggatgaaattgaatatttgactggtttactgaaaaaggttgaagaaagtaacaatgccaagaaagctcgaattgaactcctgaagaaagaggaggatttaactgctgcaagagatgtcctcaataagatgacgcagtggaactctgcactcgtggagaacattaacccttgaatctgtgtgatgacgattcattggcgtcaaaggacatctttgtataggggtgtgttagattggatgacatgtgaatgtgattcatggatcagcctggatgttctgttatgattaagaactgctatgtatc

SEQ ID NO: 20 - DNA, 1374 bp, Zea mays
atggcgagctccttctccttcccgctgatgaatccggcggacattgcggaggcgctgcacagctactgcatcgcgcccagccccggcctccgcgccgagcacatcgtcaccccgcagcccggcctcgtcgccgaggtcctcgccctcttcctcgccaacttcgtcggcgacgacgagcccgacgagcagctagagtttcaggcgctgcaggcgatcgacaacccggagcaccacatgcgcgccatagcgctctcgcgcctctaccggagggccaacgccttcctccactccatcctcttccgggacctcacgctccgcgaccttctccgcgccgacggccgccgtgtcgtctacatcctcagcgccataatcaacttcctccactacaggcaagataagctcagctttcttgagcccatcgtccaagagtacagcccccttcaggaacggcacacggagctcagggctaagatcgccgagcttcggaacgcaaaagaagaccatctacttaaggagcagatggaggcgcccgtggtccagcaactcgagaaggaggtcaatgccctcaaacagaggcttcgtgaatacaacaaagagcagctgtcgctgcgcgttgcaacaaaagccatagacgaaaagaaacaggagttactcggcaagatgaaccaagctgatttcgagttggtgaaagttatgcaagaaaaggaaaggctatcagccaaaattgttcattcccctgaaaagctcctgaggacccttgaggagaagaaagcagtccgtgatgagctaaggaactcagagaaggtggcaatgcaaaaagttcaagagaaaaccaatactcttgagacgtatactaaggtttctgagaaattagtgaagcatctctctaaaatttctgctgtacatgaaaaaggcactgttgctaaagcttctgagaaggatgttaaagcacacaaagagaagattggtgatcaaaatttggagataaaggcacttcgcaataaagctgctgagtggcaattgaaagtactcgagaacgagggcattctgaacgcaaaggagaaagaaagggatcaaagagttgcagagaacaatctgaagatgaacgcgttgaaatctgaagtcgaatcggagcacaagtgccttgaggagaaagaaataaaaataaaggaaaagcttgaaaaggttgctgaattatgctctcaagctgattcggttgctgaagctggaaataagaaaatacaggaaatctgtgggaagtataatcaagtttgcgaggctgcaaacatgtacatggatggcatcgatcgttcctttgaagagatcgatgaagctgcagtgagtttgaaagctataggccagagtggtgcttga

(19) United States
(12) Patent Application Publication
(10) Pub. No.: US 2013/0031671 A1
(43) Pub. Date: Jan. 31, 2013

(54) PLANT ARTIFICIAL CHROMOSOMES AND METHODS OF MAKING THE SAME

(71) Applicant: R. Kelly Dawe, Athens, GA (US)

(72) Inventor: R. Kelly Dawe, Athens, GA (US)

(73) Assignee: UNIVERSITY OF GEORGIA RESEARCH FOUNDATION, INC., Athens, GA (US)

(21) Appl. No.: 13/393,517

(22) PCT Filed: Aug. 31, 2010
(86) PCT No.: PCT/US10/47400

§ 371 Date: Sep. 28, 2012

Related U.S. Application Data
(60) Provisional application No. 61/238,591, filed on Aug. 31, 2009.
(60) Provisional application No. 61/238,561, filed on Aug. 31, 2009.
(60) Provisional application No. 61/275,847, filed on Sep. 3, 2009.

(51) Int. Cl. C12N 15/82; A01H 5/00; A01H 5/10; C12N 15/11

(52) U.S. Cl. 800/298; 536/23.1; 435/320.1; 435/468

(57) ABSTRACT

An engineered centromere, and systems and methods of using the engineered centromere are described. The engineered centromere can have tandem repeats of a DNA sequence with binding motifs to permit binding of fusion proteins that include a DNA binding protein and a kinetochore protein to activate the engineered centromere. Also described are a plant artificial chromosome that includes the engineered centromere, a transgenic plant containing the engineered chromosome, and a method of synthesizing a large molecule by adding multiple genes using the plant artificial chromosome.

### CROSS REFERENCE TO RELATED APPLICATION

This application claims the benefit of U.S. Provisional Application No. 61/238,561, filed Aug. 31, 2009, entitled “PLANT ARTIFICIAL CHROMOSOMES AND METHODS OF MAKING THE SAME;” U.S. Provisional Application No. 61/238,591, filed Aug. 31, 2009, entitled “PLANT ARTIFICIAL CHROMOSOMES AND METHODS OF MAKING THE SAME;” and U.S. Provisional Application No. 61/275,847, filed Sep. 3, 2009, entitled “PLANT ARTIFICIAL CHROMOSOMES AND METHODS OF MAKING THE SAME.” Each application is incorporated herein in its entirety by reference as if fully set forth herein.

### STATEMENT REGARDING FEDERALLY SPONSORED RESEARCH OR DEVELOPMENT

This invention was made in part with U.S. government support under National Science Foundation (NSF) Grant #0421671. The U.S. government has certain rights in the invention.

### FIELD OF THE INVENTION

The field of invention relates to genetic transformation. In particular, the invention concerns and embodies the synthesis and use of an artificial chromosome (AC) for transformation in plants and large molecule synthesis.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

### Brief Introduction to Plant Artificial Chromosomes

Plant artificial chromosomes are widely viewed as the future of transformation vectors for crop improvement. In principle they can circumvent many of the major problems associated with preparing transgenic crops by TDNA transformation. Namely, on an artificial chromosome, new genes will not be inserted into the genome where they can cause new mutations, new genes will have a consistent genetic context so that their expression is more uniform, and instead of adding one gene at a time, many genes can be added at once.

An artificial chromosome generally has three parts - a centromere, a gene cassette, and telomeres such that the entire artificial chromosome transmits through mitosis and meiosis normally.

A challenging feature of any artificial chromosome is the centromere. Centromeres are very large and do not have consistent sequence features that can be used to assure activation. The two existing artificial chromosome methods follow the “top down” or “bottom up” strategies for employing centromeres. In the top down method, a chromosome is whittled down by telomere truncation, and site specific recombination sites are added to the new smaller chromosome. In the “bottom up” strategy, known centromeres sequences are cloned into a vector that is ultimately treated much like a plasmid. A limitation of both methods is that they rely on natural centromeres, which are inherently unstable at several levels. The top down method produced chromosomes that were poorly transmitted (Yu et al. Proc Natl Acad Sci U S A 104(21): 8924-9 (2007)) and the bottom up strategies appear to be unpredictable and are viewed with skepticism (Ananiev et al. Chromosoma 118(2):157-77 (2009); Carlson et al. PLoS Genet 3(10):1965-74 (2007)). Perhaps most problematic feature of both the top down and bottom up strategies is that the sequence of the vector cannot be known with certainty.

### SUMMARY

Some embodiments include an engineered centromere with tandem repeats of a DNA sequence, which can contain one or more binding motifs for one or more DNA binding proteins, wherein the one or more binding motifs permit binding of one or more fusion proteins that contains the DNA binding protein and a kinetochore protein to activate the engineered centromere. The fusion protein can further include a nuclear localization signal, such as, for example, a nuclear localization signal to PKKRKV. The fusion protein can further include an eptitope recognition sequence. The epitope recognition sequence can include, but is not limited to, multimers of the HA epitope tag YPYDVPDYA.

In some embodiments, the DNA sequence can have one or more binding motifs for one or more DNA binding proteins. Some embodiments include a DNA sequence with DNA binding motifs TetR (SEQ ID NO. 1), CENP-B box (SEQ ID NO. 2), LacO (SEQ ID NO. 3), LexA (SEQ ID NO. 4), or Gal4 (SEQ ID NO. 5). Some embodiments include a DNA sequence with combinations of DNA binding motifs TetR (SEQ ID NO. 1), CENP-B box (SEQ ID NO. 2), LacO (SEQ ID NO. 3), LexA (SEQ ID NO. 4), or Gal4 (SEQ ID NO. 5). The DNA sequence can have filler nucleic acid residues between each of the one or more binding motifs. The filler nucleic acid residues can be, but are not limited to, about 5-50 bp in length, or 50 bp or longer. Some embodiments include a DNA molecule with tandem repeats of the DNA sequence having one or more binding motifs for one or more DNA binding proteins.

Some embodiments include an engineered centromere with tandem repeats of a DNA sequence as set forth in SEQ ID NO. 6.

In some embodiments, the engineered centromere can have at least 500 tandem repeats. In other embodiments, DNA molecule can have at least 1000 tandem repeats. In some embodiments, the DNA binding proteins can include Lad, LexA, Gal4, TetR, CENP-B, or fragments thereof. In other embodiments, DNA binding proteins can be combinations of Lad, LexA, Gal4, TetR, CENP-B, and fragments thereof. In some embodiments, one or more kinetochore proteins can be fused with one or more DNA binding proteins. In certain embodiments, the one or more DNA binding proteins can be a polypeptide encoded by SEQ ID. NO. 7, amino acids 1-72 of a polypeptide encoded by SEQ ID NO. 8, amino acids 1-74 of a polypeptide encoded by SEQ ID NO. 9, amino acids 1-206 of a polypeptide encoded by SEQ ID NO. 10, amino acids 1-205 of a polypeptide encoded by SEQ ID NO. 11, or combinations thereof. In some embodiments, one or more kinetochore proteins can be CENH3, CENP-C, MIS12, CENP-H, CENP-O/MCM21, NDC80, SPC24, CENP-A/CENH3, CENP-S, CENP-T, NNF1, NUF2, SPC25, fragments thereof, or combinations thereof

Some embodiments include a method of activating an artificial centromere by providing an artificial centromere and contacting the artificial centromere with one or more fusion proteins. The fusion protein or fusion proteins can include one or more DNA binding proteins and one or more kinetochore proteins, whereby the DNA binding protein portion of one or more fusion proteins can bind to the artificial centromere and a kinetochore is formed.

Some embodiments include a plant artificial chromosome (AC) including the engineered centromere.

Some embodiments include a transgenic plant with an artificial chromosome (AC) that includes the engineered centromere. In some embodiments, the transgenic plant AC can express one or more fusion proteins that can include one or more DNA binding proteins and one or more kinetochore proteins. In some embodiments, the transgenic plant AC can include a nucleic acid molecule capable of expressing one or more fusion proteins, which can include one or more DNA binding proteins and one or more kinetochore proteins. Some embodiments include a seed carrying the artificial chromosome that includes the engineered centromere.

Some embodiments include a system that includes an engineered centromere, which includes tandem repeats of a DNA sequence with one or more binding motifs for one or more DNA binding proteins and one or more filler nucleic acid residues between each of the one or more binding motifs, as well as one or more nucleic acids expressing one or more fusion proteins that includes one or more DNA binding proteins and one or more kinetochore proteins. The one or more binding motifs can permit binding of the one or more fusion proteins to activate the engineered centromere to form a kinetochore. The fusion protein can further include a nuclear localization signal, such as, for example, a nuclear localization signal to PKKRKV. The fusion protein can further include an eptitope recognition sequence. The epitope recognition sequence can include, but is not limited to, multimers of the HA epitope tag YPYDVPDYA.

Some embodiments include a system that includes a DNA sequence with one or more binding motifs for one or more DNA binding proteins. The DNA binding motifs can be, but are not limited to, TetR (SEQ ID NO. 1), CENP-B box (SEQ ID NO. 2), LacO (SEQ ID NO. 3), LexA (SEQ ID NO. 4), or Gal4 (SEQ ID NO. 5). The DNA binding motifs can be combinations of DNA binding motifs TetR (SEQ ID NO. 1), CENP-B box (SEQ ID NO. 2), LacO (SEQ ID NO. 3), LexA (SEQ ID NO. 4), or Gal4 (SEQ ID NO. 5). The DNA sequence can have filler nucleic acid residues between each of the one or more binding motifs. The filler nucleic acid residues can be, but are not limited to, about 5-50 bp in length, or 50 bp or longer. Some embodiments include a DNA molecule with tandem repeats of the DNA sequence having one or more binding motifs for one or more DNA binding proteins.

In some embodiments, the system includes an engineered centromere with tandem repeats of a DNA sequence as set forth in SEQ ID NO. 6.

In some embodiments, the engineered centromere can have at least 500 tandem repeats. In other embodiments, the engineered centromere can have at least 1000 tandem repeats. In some embodiments, the DNA binding proteins can include Lad, LexA, Gal4, TetR, CENP-B, or fragments thereof. In other embodiments, the DNA binding proteins can be combinations of LacI, LexA, Gal4, TetR, CENP-B, and fragments thereof. In some embodiments, one or more kinetochore proteins can be fused with one or more DNA binding proteins. In certain embodiments, the one or more DNA binding proteins can be a polypeptide encoded by SEQ ID. NO. 7, amino acids 1-72 of a polypeptide encoded by SEQ ID NO. 8, amino acids 1-74 of a polypeptide encoded by SEQ ID NO. 9, amino acids 1-206 of a polypeptide encoded by SEQ ID NO. 10, amino acids 1-205 of a polypeptide encoded by SEQ ID NO. 11, or combinations thereof. In some embodiments, one or more kinetochore proteins can be CENH3, CENP-C, MIS12, CENP-H, CENP-O/MCM21, NDC80, SPC24, CENP-A/CENH3, CENP-S, CENP-T, NNF1, NUF2, SPC25, fragments thereof, or combinations thereof.

Some embodiments include a method of synthesizing a large molecule by adding multiple genes using the plant artificial chromosome. In some embodiments, an artificial chromosome can be synthesized, one or more recruiting constructs can be introduced, and the transformed artificial chromosome can be activated by co-expressing one or more fusion proteins that includes one or more DNA binding proteins and one or more kinetochore proteins. In some embodiments, the artificial chromosome can be synthesized by full gene synthesis.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1 depicts, in accordance with an embodiment herein, production of Arrayed Binding Sites (ABS) arrays, their successful transformation into maize, and demonstration that they recruit the DNA binding protein Lad fused with a fluorescent tag.

A) The structure of ABS arrays. Three consecutive monomers are shown. Each monomer contains the binding sites for LacI, LexA and Gal4.

B) Production of ABS arrays using overlapping primers.

C) ABS PCR products do not enter an agarose gel and digest with Ndel.

D) Assays of two ABS maize lines by Southern blotting. HindIII does not cut in the array, while Ndel does. ABS-ch3 has the longest arrays; ABS-ch7 has the smallest. The arrays are tandem and continuous.

E) FISH analysis of ABS-ch7 at pachytene. A single bright insertion point is detected (arrow 1). The green spot close by (arrow 2) shows the centromere on chromosome 7.

F) FISH analysis of ABS-ch3 at mitotic metaphase. There is a single insertion mid-arm on chromosome 3L. The signal from the red ABS locus (boxed area) is brighter than the green signal detected from the major centromere repeats CentC.

G) Demonstration that ABS recruits Lad. A LacI-YFP protein fluoresces brightly when tethered at the ABS-ch3 locus (arrows).

### DETAILED DESCRIPTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3^{rd} ed., J. Wiley & Sons (New York, N.Y. 2001); March, Advanced Organic Chemistry Reactions, Mechanisms and Structure 5^{th} ed., J. Wiley & Sons (New York, N.Y. 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

With the benefit of the present disclosure, one skilled in the art will appreciate many methods and materials similar or equivalent to those described herein, which could be used in the practice of the present invention. Indeed, the present invention is in no way limited only to those methods, materials, applications, and objects of application that are specifically described herein.

The Kinetochore Tethering Concept

This disclosure relates to a way to design artificial chromosome vectors. Instead of relying on existing centromeres, an entirely synthetic system is employed that circumvents the instability of centromeres by enforcing by a genetic determination process. It is a two component system containing engineered centromeres as well as proteins that are designed to activate the centromeres. The engineered centromeres contain long arrays of repeats with known DNA binding motifs. Examples of the DNA binding motifs are listed in Table 1. The activating proteins are key kinetochore proteins that have been or can be fused to the DNA binding proteins that bind to the synthetic centromeres. The tethered proteins, either alone or in combination, recruit the rest of the kinetochore and support chromosome segregation. The DNA binding protein(s) (also referred to herein as a binding module) can be, but are not limited to, proteins listed in Table 2. The kinetochore proteins can be, but are not limited to, those listed in Table 3A and Table 3B. In principle, any DNA binding module that binds to a known motif, from any species, can be used in this manner.

TABLE 1
DNA binding motifs.

| DNA binding motif | SEQ ID NO. |
|---|---|
| TetR (19) | 1 |
| TCCCTATCAGTGATAGAGA | |
| CENP-B box (17) | 2 |
| TTTCGTTGGAAACGGGA | |
| LacO (21) | 3 |
| AATTGTGAGCGGCTCACAATT | |
| LexA (20) | 4 |
| TACTGTATATATATACAGTA | |
| Gal4 (17) | 5 |
| CGGAGGACTGTCCTCCG | |

TABLE 2
DNA binding proteins.

| Protein | Accession No. | SEQ ID NO. | Binding region |
|---|---|---|---|
| LacI | AAA24052 | 7 | Whole protein |
| LexA | ZP_06936566 | 8 | Amino acids 1-72 |
| | | | of a polypeptide |
| | | | encoded by SEQ ID |
| | | | NO. 8 |
| Gal4 | CAA97969 | 9 | Amino acids 1-74 |
| | | | of a polypeptide |
| | | | encoded by SEQ ID |
| | | | NO. 9 |
| TetR | CAA32196 | 10 | Amino acids 1-206 |
| | | | of a polypeptide |
| | | | encoded by SEQ ID |
| | | | NO. 10 |
| CENP-B | AAH53847 | 11 | Amino acids 1-125 |
| | | | of a polypeptide |
| | | | encoded by SEQ ID |
| | | | NO. 11 |

The DNA binding modules can be, but are not limited to, Lad, LexA, TetR, Gal4, or CENP-B. The DNA binding modules can be derived from, for example, E. coli, human, yeast, or other species. In some embodiments, the protein sequences of the DNA binding modules are preserved, and the encoding DNA sequences are changed to reflect the optimum codon usage for maize. Since prokaryotes lack a nuclear envelope, a nuclear localization signal can be added to the fusion proteins to assure that the proteins can be imported into plant nuclei. In some embodiments, the nuclear localization signal can be to PKKKRKV or others. In some embodiments, epitope recognition sequences can be added. The epitope recognition sequence can be, but is not limited to, multimers of the HA epitope tag YPYDVPDYA.

In some embodiments, modified forms and/or variants of the above sequences and those listed in Table 1 and Table 2 can be used, wherein the modifications and/or variants can include length modifications. The numbers of nucleic acids for the binding motifs can be at least 10, or at least 11, or at least 12, or at least 13, or at least 14, or at least 15, or at least 16, or at least 17, or at least 18, or at least 19, or at least 20, or at least 21, or at least 22, or at least 23, or at least 24 or at least 25, or more. The numbers of amino acids of the binding protein can be at least 20, or at least 30, or at least 40, or at least 50, or at least 60, or at least 70, or at least 80, or at least 90, or at least 100, or at least 110, or at least 120, or at least 130, or at least 140, or at least 150, or at least 160, or at least 170, or at least 180, or at least 190, or at least 200 or more. The residue variations can be, for example, conservative substitutions, common substitutions, and others. The modified forms and variants can be naturally occurring variants, e.g., from other species.

TABLE 3A
Kinetochore proteins-encoded by the SEQ ID NO.
as indicated

| Protein | Accession No. | SEQ ID NO. |
|---|---|---|
| CENH3/CENP-A | AF519807 | 12 |
| CENP-C | AF129857 | 13 |
| MIS12 | FJ971487 | 14 |
| CENP-o/MCM21 | BT024183 | 15 |
| NDC80 | EU971283 | 16 |
| CENP-S | EU966192 | 17 |
| CENP-T | BT041097 | 18 |
| NNF1 | EC890639 | 19 |
| NUF2 | BT040808 | 20 |
| SPC25 | (predicted gene, | |
| | maizegdb.org) | |

TABLE 3B
Human kinetochore proteins and their likely homologues (Cheeseman
and Desai, Mol Cell Biol (2008)).

| | | Alternate | | | | | | |
|---|---|---|---|---|---|---|---|---|
| Complex | Human | names | Accession No. | S. pombe | S. cerevisiae | C. elegans | D. melanogaster | A. thaliana |
| | CENP-A | CENH3 | AF519807 | Cnp1 | Cse4 | HCP-3/ | CID | CENH3/ |
| | | | | | | CENP-A | | HTR12 |
| | CENP-B | | | Abp1, Cbh1, | | | | |
| | | | | Cbh2 | | | | |
| CCAN | CENP-C | | AF129857 | Cnp3 | Mif2 | HCP-4/ | Cenp-C/ | CENP-C |
| | | | | | | CENP-C | CG31258 | |
| CCAN | CENP-H | | | Fta3 | Mcm16 | | | |
| CCAN | CENP-I | MIS6; | | Mis6 | Ctf3 | | | |
| | | LRPR1 | | | | | | |
| CCAN | CENP-K | Solt; AF- | | Sim4 | | | | |
| | | 5a; | | | | | | |
| | | FKSG14; | | | | | | |
| | | ICEN37 | | | | | | |
| CCAN | CENP-50 | CENP-U; | | | | | | |
| | | MLF1IP; | | | | | | |
| | | PBIP1; | | | | | | |
| | | ICEN24 | | | | | | |
| CCAN | CENP-0 | MCM21R; | BT024183 | Mal2 | Mcm21 | | | |
| | | MGC11266; | | | | | | |
| | | ICEN36 | | | | | | |
| CCAN | CENP-P | LOC401541 | | Fta2 | Ctf19 | | | |
| CCAN | CENP-Q | FLJ10545 | | Mis17 | | | | |
| CCAN | CENP-R | ITGB3BP | | | | | | |
| CCAN | CENP-L | FTA1R; | | Fta1 | | | | |
| | | dJ383J4.3; | | | | | | |
| | | ICEN33 | | | | | | |
| CCAN | CENP-M | PANE1; | | | | | | |
| | | ICEN39 | | | | | | |
| CCAN | CENP-N | Chl4R; | | Mis15 | Chl4 | | | |
| | | BM039 | | | | | | |
| CCAN | CENP-T | FLJ1311; | BT041097 | | | | | |
| | | ICEN22 | | | | | | |
| CCAN | CENP-S | | EU966192 | | | | | |
| Mis18 | MIS18a | C21orf45 | | Mis18 | | | | |
| complex | | | | | | | | |
| Mis18 | MIS18P | Opa- | | Mis18 | | | | |
| complex | | interacting | | | | | | |
| | | protein 5 | | | | | | |
| Mis18 | KNL2 | M18BP1; | | | | KNL-2 | | |
| complex | | C14orf106 | | | | | | |
| Mis12 | MIS12 | | FJ971487 | Mis12 | Mtw1 | MIS-12 | CG18156 | Mis12 |
| complex | | | | | | | | |
| Mis12 | DSN1 | Q9H410; | | Mis13/Dsn1 | Dsn1 | KNL-3 | | |
| complex | | C20orf1 72 | | | | | | |
| Mis12 | NNF1 | PMF1 | EC890639 | Nnf1 | Nnf1 | KBP-1 | CG13434/CG31658 | |
| complex | | | | | | | | |
| Mis12 | NSL1 | DC31 | | Mis14/Nsl1 | NsM | KBP-2 | CG1558 | |
| complex | | | | | | | | |
| Ndc80 | NDC80 | HEC1 | EU971283 | Ndc80 | Ndc80 | NDC-80 | CG9938-PA | |
| complex | | | | | | | | |
| Ndc80 | NUF2 | | BT040808 | Nuf2 | Nuf2 | HIM-10 | CG8902 | Nuf2 |
| complex | | | | | | | | |
| Ndc80 | SPC24 | | | Spc24 | Spc24 | KBP-4 | CG7242 | |
| complex | | | | | | | | |
| Ndc80 | SPC25 | | (predicted gene, | Spc25 | Spc25 | KBP-3 | | |
| complex | | | maizegdb.org) | | | | | |
| | KNL1 | AF15q14; | | Spc7 | Spc105 | KNL-1 | CG11451 | |
| | | CASC5; | | | | | | |
| | | D40 | | | | | | |
| | Zwint | | | | | KBP-5? | | |
| RZZ | ROD | | | — | — | ROD-1 | ROD | |
| complex | | | | | | | | |
| RZZ | ZW10 | | | — | — | CZW-1 | ZW10 | ZW10 |
| complex | | | | | | | | |
| RZZ | Zwilch | | | — | — | ZWL-1 | Zwilch | |
| complex | | | | | | | | |
| | CENP-F | Mitosin | | | | HCP-1/2? | | |
| | Spindly | Coiled-coil | | | | C06A8.5 | Spindly/CG15415 | |
| | | domain- | | | | | | |
| | | containing | | | | | | |
| | | 99 | | | | | | |
| | Dynein | | | | Not at | DHC-1 | Several DHCs | Absent |
| | | | | | kinetochores | | | |
| | NDE1 | | | | | NUD-2 | | |
| | NDEL1 | | | | | NUD-2 | | |
| | NUDC | | | | | NUD-1 | | |
| | LIS1 | | | | | LIS-1 | | |
| | SKA1 | C18orf24 | | | | Y106G6H.1 5 | | AT3G60660 |
| | SKA2 | Fam33A | | | | | | |
| | CLASP1, | | | Peg1 | Stu1 | CLS-2 | MAST/Orbit | |
| | CLASP2 | | | | | | | |
| | CLIP170 | Restin | | Tip1 | Bik1 | M01A8.2 | | |
| | EB1 | | | Mal3 | Bim1 | EBP-1 and | | |
| | | | | | | EBP-2 | | |
| | TOG | XMAP215 | | Dis1, Alp14 | Stu2 | ZYG-9 | Msps | MORI |
| | Kif2A, Kif2B, | Kinesin-13; | | | | KLP-7 | KLP10A, | KINESIN- |
| | Kif2C/MCAK | XKCM1 | | | | | KLP59C | 13A; MSL1.9 |
| | ICIS | | | | | | | |
| | KIF18A | Kinesin-8 | | Klp5/6 | Kip3 | KLP-13 | KLP67A | |
| | CENP-E | | | — | — | — | CENP- | |
| | | | | | | | meta/CENP- | |
| | | | | | | | ana | |
| Mitotic | MAD1 | | | Mad1 | Mad1 | MDF-1 | | |
| checkpoint | | | | | | | | |
| Mitotic | BUB1 | | | Bub1 | Bub1 | BUB-1 | Bub1 | |
| checkpoint | | | | | | | | |
| Mitotic | BUB3 | | | Bub3 | Bub3 | BUB-3 | Bub3 | |
| checkpoint | | | | | | | | |
| Mitotic | BUBR1 | | | Mad3 | Mad3 | SAN-1 | BubR1 | |
| checkpoint | | | | | | | | |
| Mitotic | MAD2 | | | Mad2 | Mad2 | MDF-2 | Mad2 | |
| checkpoint | | | | | | | | |
| Mitotic | CDC20 | | | Slp1 | Cdc20 | FZY-1 | Fzy | |
| checkpoint | | | | | | | | |
| Mitotic | MPS1 | TTK | | Mph1 | Mps1 | — | Mpsl/ald | |
| checkpoint | | | | | | | | |
| Mitotic | PICH | FLJ20105 | | — | — | — | — | AT5G63950 |
| checkpoint | | | | | | | | |
| Mitotic | TA01 | MARKK | | | | | | |
| checkpoint | | | | | | | | |
| Chromosome | Aurora B | | Ark1 | IpM | AIR-2 | Aurora B | | |
| passenger | | | | | | | | |
| complex | | | | | | | | |
| Chromosome | INCENP | | | Plc1 | SN15 | ICP-1 | INCENP | |
| passenger | | | | | | | | |
| complex | | | | | | | | |
| Chromosome | Survivin | | | Bir1/Cut7 | Bir1 | BIR-1 | | |
| passenger | | | | | | | | |
| complex | | | | | | | | |
| Chromosome | Borealin | Dasra | | | | CSC-1 | | |
| passenger | | | | | | | | |
| complex | | | | | | | | |
| | TD60 | | | | | | | |
| | SG01 | SGOL1 | | Sgo1 | Sgo1 | C33H5.15 | MEI-S332 | AT3G 10440.1 |
| | SG02 | SGOL2/TR | | Sgo2 | | | | AT5G04420.1 |
| | | IPI N | | | | | | |
| | PP2A | | | | | PPH-5 | | |
| | PPlγ | | | | Glc7 | GSP-1/2 | | |
| | Polo-like | PLK1 | | Plo1 | Cdc5 | PLK-1 | Polo | |
| | kinase 1 | | | | | | | |
| Nup107-160 | NUP107 | | | Not at | Not at | NPP-5 | Nup107 | |
| complex | | | | kinetochores | Kinetochores | | | |
| Nup107-160 | NUP85 | | | | | NPP-2 | | |
| complex | | | | | | | | |
| Nup107-160 | NUP133 | | | | | NPP-15 | | |
| complex | | | | | | | | |
| Nup107-160 | NUP160 | | | | | NPP-6 | | |
| complex | | | | | | | | |
| Nup107-160 | NUP96 | | | | | NPP-10 | | |
| complex | | | | | | | | |
| Nup107-160 | NUP120 | | | | | | | |
| complex | | | | | | | | |
| Nup107-160 | Nup37 | | | | | | | |
| complex | | | | | | | | |
| Nup107-160 | NUP43 | | | | | | | |
| complex | | | | | | | | |
| Nup107-160 | SEC13 | | | | | NPP-20 | | |
| complex | | | | | | | | |
| Nup107-160 | SEH1 | | | | | NPP-18 | | |
| complex | | | | | | | | |
| | ELYS | | | | | MEL-28 | | |
| | CRM1 | | | CRM1 | | IMB-4 | emb | |
| | RanBP2 | NUP358 | | | | NPP-9 | | |
| | RanGAPI | | | | | RAN-2 | | |

In some embodiments, modified forms and/or variants of the polypeptide or protein encoded by the above sequences and those listed in Table 3A and Table 3B can be used, wherein the modifications and/or variants can include length modifications. The numbers of amino acids can at least 20, or at least 30, or at least 40, or at least 60, or at least 100, or at least 200, or at least 300, or at least 400, or at least 500, or at least 600, or at least 700, or at least 800, or at least 900, or at least 1000, or at least 1200, or at least 1400, or at least 1600, or at least 1800, or at least 2000 or more. The residue variations can be, for example, conservative substitutions, common substitutions, and others. The modified forms and variants can be naturally occurring variants, e.g., from other species.

Centromere

Some embodiments of the present invention provide for a DNA sequence comprising binding motifs for one or more DNA binding proteins (also referred to herein as binding module). The binding motifs are regions of the DNA wherein DNA binding proteins will bind. The binding motifs can also be referred to throughout this specification as a DNA binding site. In certain embodiments, the one or more DNA binding motifs can be selected from the group consisting of TetR (SEQ ID NO. 1), CENP-B box (SEQ ID NO. 2), LacO (SEQ ID NO. 3), LexA (SEQ ID NO. 4), Gal4 (SEQ ID NO. 5), and combinations thereof.

In certain embodiments, the DNA sequence comprises filler nucleic acid residues between each of the binding sites. In various embodiments, the filler nucleic acid residues can be, but are not limited to, 50 bp or longer. In other embodiments, the filler nucleic acid residues are about 5-50 bp in length. In other embodiments, the filler nucleic acid residues are about 5, 10, 15, 20, 25, 30, 35, 40 or 50 bp in length. In still other embodiments, the filler nucleic acid residues are about 12 to 13 bp in length.

In certain embodiments the DNA sequence can be SEQ ID NO. 6. In other embodiments, the DNA sequence can be 160 bp to 180 bp. In other embodiments, the size of the DNA sequence can be fractions or multiples of 157 bp. The number of base pairs, 157 bp, is the single wrap of a nucleosome, and the size of the maize centromeric repeat.

In some embodiments, the number of base pairs can be fractions or multiple of the number of base pairs corresponding to the centromeric repeat length of a selected species other than maize.

Some embodiments of the present invention provide for a DNA molecule comprising tandem repeats of a DNA sequence comprising binding motifs for one or more DNA binding proteins. In some embodiments the DNA molecule comprises at least 100, 200, 300, 400, 500, 600, 700, 800, 900, 1000, 1100, 1200, 1300, 1400, or 1500 tandem repeats of a DNA sequence comprising binding motifs for one or more DNA binding proteins. In some embodiments the DNA molecule comprises at least 500 tandem repeats of a DNA sequence comprising binding motifs for one or more DNA binding proteins. In some embodiments the DNA molecule comprises at least 1000 tandem repeats of a DNA sequence comprising binding motifs for one or more DNA binding proteins. In certain embodiments, the one or more DNA binding motifs can be selected from the group consisting of TetR (SEQ ID NO. 1), CENP-B box (SEQ ID NO. 2), LacO (SEQ ID NO. 3), LexA (SEQ ID NO. 4), Gal4 (SEQ ID NO. 5), and combinations thereof.

In certain embodiments, the DNA sequence comprising binding motifs for one or more DNA binding proteins is SEQ ID NO. 6. Thus, in certain embodiments, the DNA molecule comprises tandem repeats of SEQ ID NO. 6.

Some embodiments disclosed herein relate to an artificial centromere. In various embodiments, the DNA molecule comprising tandem repeats of a DNA sequence comprising binding motifs for one or more DNA binding proteins is the artificial centromere.

Some embodiments described herein provide for a method of activating an artificial centromere. The method can comprise providing an artificial centromere described herein, and combining the artificial centromere with one or more fusion proteins comprising one or more DNA binding proteins and one or more kinetochore proteins, whereby the DNA binding protein portion of the one or more fusion proteins binds to the artificial centromere and a kinetochore is formed. Key inner kinetochore proteins such as, for example, CENH3 and CENPC are required to recruit all other proteins in the mature kinetochores, inasmuch as when one such protein is absent, all other kinetochore proteins fail to localize. The system as described is designed to accommodate the full complexity of the kinetochore formation process. Since the scaffold (i.e., DNA sequence with binding motifs) supports multiple binding sites (i.e. binding motifs), the kinetochore recruitment process can be tailored and optimized.

In some embodiments, the one or more DNA binding proteins can be selected from Table 2. In certain embodiments, the one or more kinetochore proteins can be selected from Table 3A and Table 3B. In certain embodiments, the fusion protein can be configured for the DNA binding protein to bind with the centromere.

Some embodiments include a system that includes an engineered centromere, which includes tandem repeats of a DNA sequence with one or more binding motifs for one or more DNA binding proteins and one or more filler nucleic acid residues between each of the one or more binding motifs, as well as one or more nucleic acids expressing one or more fusion proteins that includes one or more DNA binding proteins and one or more kinetochore proteins. The one or more binding motifs can permit binding of the one or more fusion proteins to activate the engineered centromere to form a kinetochore. The fusion protein can further include a nuclear localization signal such as, for example, a nuclear localization signal to PKKRKV. The fusion protein can further include an eptitope recognition sequence. The epitope recognition sequence can include, but is not limited to, multimers of the HA epitope tag YPYDVPDYA.

Some embodiments include a system that includes a DNA sequence with one or more binding motifs for one or more DNA binding proteins. The DNA binding motifs can be, but are not limited to, SEQ ID NO. 1, SEQ ID NO. 2, SEQ ID NO. 3, SEQ ID NO. 4, or SEQ ID NO. 5. The DNA binding motifs can be combinations of DNA binding motifs TetR (SEQ ID NO. 1), or combinations thereof. The DNA sequence can have filler nucleic acid residues between each of the one or more binding motifs. The filler nucleic acid residues can be, but are not limited to, about 5-50 bp in length, or 50 bp or longer.

In some embodiments, the system includes an engineered centromere with tandem repeats of a DNA sequence as set forth in SEQ ID NO. 6.

In some embodiments, the system includes an engineered centromere with at least 500 tandem repeats. In other embodiments, the system can include an engineered centromere with at least 1000 tandem repeats. In some embodiments, the system can have DNA binding proteins such as, for example, LacI, LexA, Gal4, TetR, CENP-B, or fragments thereof. In other embodiments, the DNA binding proteins in the system can be combinations of LacI, LexA, Gal4, TetR, CENP-B, and fragments thereof. In some embodiments, one or more kinetochore proteins in the system can be fused with one or more DNA binding proteins. In certain embodiments, the one or more DNA binding proteins of the system can be a polypeptide encoded by SEQ ID. NO. 7, amino acids 1-72 of a polypeptide encoded by SEQ ID NO. 8, amino acids 1-74 of a polypeptide encoded by SEQ ID NO. 9, amino acids 1-206 of a polypeptide encoded by SEQ ID NO. 10, amino acids 1-205 of a polypeptide encoded by SEQ ID NO. 11, or combinations thereof. In some embodiments, one or more kinetochore proteins in the system can be CENH3, CENP-C, MIS12, CENP-H, CENP-O/MCM21, NDC80, SPC24, CENP-A/CENH3, CENP-S, CENP-T, NNF1, NUF2, SPC25, fragments thereof, or combinations thereof

Some embodiments include a method of synthesizing a large molecule by adding multiple genes using the plant artificial chromosome. In some embodiments, an artificial chromosome can be synthesized, one or more recruiting constructs can be introduced, and the transformed artificial chromosome can be activated by co-expressing one or more fusion proteins that includes one or more DNA binding proteins and one or more kinetochore proteins. In some embodiments, the artificial chromosome can be synthesized by full gene synthesis.

Some embodiments disclosed herein relate to the method of creating artificial centromeres. Some embodiments relate to creating sequences that contain binding sites for DNA binding proteins, and amplifying the sequences into Arrayed Binding Sites (ABS). Amplification can be achieved by, for example, overlapping PCR, and other multimerization methods. As used herein, about indicates ±20% variation of the value it describes. It is understood that the specific dimensions described herein are for illustration purposes and are not intended to limit the scope of the application. Merely by way of example, the resulting PCR products can be at least about 50 kb, or at least about 75 kb, or at least about 100 kb, or at least about 125 kb, or at least about 150 kb, or at least about 175 kb, or at least about 200 kb, or at least about 225 kb, or at least about 250 kb, or at least about 275 kb, or at least about 300 kb, or at least about 350 kb, or at least about 400 kb or longer. In some embodiments, PCR products are composed exclusively of ABS arrays.

In some embodiments, metal spheres are coated with the PCR product and a marker plasmid, and maize calli are transformed. The transformation can be performed using standard biolistic methods or other methods such as Agrobacterium-mediated transformation or T-DNA. In some embodiments, the PCR products are inserted at single sites in the plant genome. In some embodiments, the plant can be maize.

In some embodiments, the engineered centromere can contain arrays of repeats with one or more DNA binding motifs of Table 1. In some embodiments, kinetochore proteins are tethered to ABS arrays via DNA binding proteins of Table 2. The kinetochore proteins can be tethered alone or in combination. The kinetochore protein complex can contain one or more proteins in Table 3A or 3B.

In some embodiments, the construct can be a tri-protein chimera containing a binding module fused to an N-terminal tail and a plant histone variant core region. The N-terminus can be replaced with a sequence that allows the use of a histone antibody. The chimeral histone can bind to the ABS sites and recruit the natural histone to form a centromeric state. The centromeric state can be stable after the tethered protein is removed by segregation. In some embodiments, for example, the construct can be a tri-protein chimera containing a Gal4 binding module fused to an oat N-terminal tail and a maize CENH3 (centromeric histone H3) histone core region. The N-terminus can be replaced with, for example, an oat sequence that allows the use of an oat CENH3 antibody. The chimeral CENH3 can bind to the ABS sites and recruit natural CENH3 to form a centromeric state. The centromeric state can be stable after the tethered protein is removed by segregation.

In some embodiments, Centromere Protein C (CENPC) can be used to recruit CENH3 to DNA using a tethering construct such as, for example, a Lac1-CENPC tethering construct. In some embodiments, Minichromosome Instability 12 (MIS12) fused with a LexA-binding module may be used in a similar manner to recruit CENH3, CENPC, or other proteins that are sufficient to nucleate kinetochores at tethered sites.

In some embodiments, combinations of two or more proteins can be used by fusing each protein to a different DNA binding module, so that crossing the transgenic lines results in combination of the proteins on the same ABS array. In some embodiments, CENH3 and CENPC can be used together to recruit the entire kinetochore complex. In some embodiments, CENH3, CENPC, and MIS 12, or combinations of these and/or other proteins can be combined at the same ABS sites to confer most kinetochore functions. Without wishing to be bound by theory, these proteins are thought to bind to the ABS and kinetochore activation is believed to be occurring.

Artificial Chromosome

Some embodiments disclosed herein provide for an artificial chromosome comprising the artificial centromere of the present invention.

Methods of producing artificial chromosomes are known in the art. See e.g. Carret al. Nat Biotech 27,1151-1162 (2009) for artificial full gene synthesis, Carlson et al. PLoS Genet 3: 1965-1974 (2007) and Ananiev et al. Chromosoma 118:157-77 (2009). Accordingly, an artificial chromosome can be prepared utilizing known methods in the art and using the artificial centromere of the present invention. In various embodiments, the artificial centromere of the present invention can be used in place of the centromeres described in the known methods of synthesizing an artificial chromosome.

Some embodiments disclosed herein provide for a method of producing an artificial chromosome comprising the artificial centromere of the present invention. In various embodiments, the method can involve incorporating tethering sites into an existing chromosome such that kinetochore formation at the tether site creates an artificial second centromere that can cause chromosome breakage and formation of a new chromosome segregated by the artificial centromere only.

In other embodiments, the method can comprise transforming a large engineered circular molecule capable of segregating independently without the need for telomeres. An artificial chromosome formed in this way can include engineered genes.

In other embodiments, the method can comprise transforming a chromosome comprising an artificial centromere, one or more genes of interest, and one or more telomeres. In other embodiments, the method can comprise the approach of designing a maize artificial chromosome with telomeres as described (Ananiev et al. Chromosoma. 118:157-77 (2007)). In other embodiments, the chromosome can be a circular artificial chromosome in maize (Carlson et al. PLoS Genet. 3: 1965-1974 (2007)). In yet other embodiments, the chromosome can be used for the general utility of maize artificial chromosomes (Carlson et al. PLoS Genet. 3: 1965-1974 (2007)).

In some embodiments, the artificial chromosome formed can be similar in structure to a natural chromosome and similar in function, such as, for example, accurate segregation through mitosis and meiosis. In some embodiments, the centromere can be the centromere of the present invention and the other components such as, for example, the genes and telomeres, can be engineered to be as similar as possible to the native components.

Transgenic Seed

Some embodiments relate to a transgenic seed carrying an artificial chromosome described herein. In various embodiments, a transgenic seed comprises an artificial chromosome comprising the artificial centromere described herein. In some embodiments, the transgenic seed further comprises nucleic acids capable of expressing the fusion proteins described herein to activate the artificial centromere.

Transgenic Plant

Some embodiments relate to a transgenic plant expressing the artificial chromosome described herein. In some embodiments, the chromosome comprises the artificial centromere described herein. In some embodiments, the transgenic plant further comprises nucleic acids capable of expressing the fusion proteins described herein to activate the artificial centromere. In some embodiments, the transgenic plant can be maize.

Some embodiments include a method of achieving crop improvement by using a plant artificial chromosome. For example, genes that improve yield qualities, confer salt tolerance, confer drought tolerance, confer insect resistance, or add other beneficial agronomic traits can be added alone or in combination to molecules containing an artificial centromere.

Embodiments of the present application are further illustrated by the following examples.

### EXAMPLES

The following non-limiting examples are provided to further illustrate embodiments of the present application. It should be appreciated by those of skill in the art that the techniques disclosed in the examples that follow represent approaches discovered by the inventors to function well in the practice of the application, and thus can be considered to constitute examples of modes for its practice. To the extent that specific materials are mentioned, it is merely for purposes of illustration and is not intended to limit the invention. Those of skill in the art should, in light of the present disclosure, appreciate that many changes can be made in the specific embodiments that are disclosed and still obtain a like or similar result without departing from the spirit and scope of the application.

### Example 1A

### Preparing an Engineered Centromere

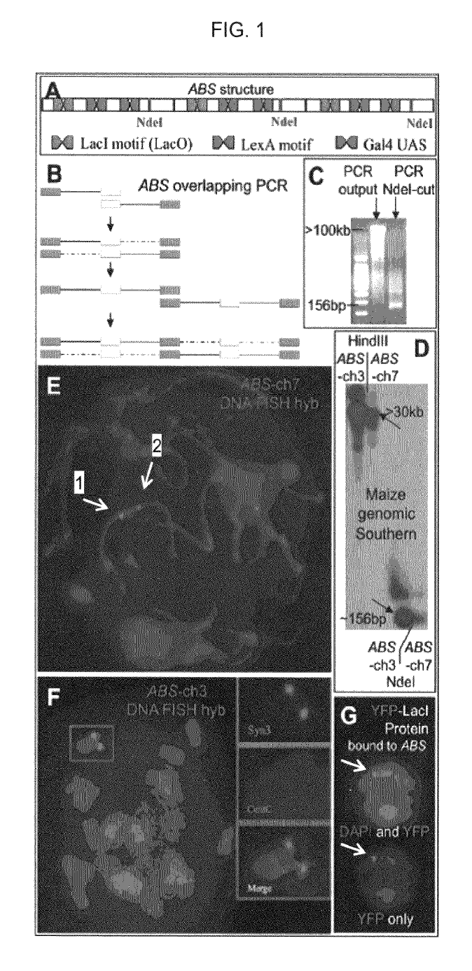

A 156 bp sequence was created that contained binding sites for four different DNA binding modules (Lacl, Gal4, LexA, and TetR), each of which are known to tether proteins in plants (Matzke et al. Plant Molecular Biology Reporter 21(1):9-19 (2003); Matzke et al. Plant Physiology 139(4): 1586-1596 (2005); Bohner et al. Plant J 19(1):87-95 (1999); Zuo et al. Current Opinion in Biotechnology 11(2): 146-151 (2000); Zuo et al. Methods Mol Biol 323: 329-42 (2006)). In order to multimerize the monomer, these were amplified into long Arrayed Binding Sites (called ABS) by overlapping PCR (FIG. 1). Long>200 kb PCR products composed exclusively of ABS arrays were created in-this way. Metal spheres were then coated with the PCR product and a marker plasmid, and maize calli were transformed by standard biolistic methods. In three resulting transgenic lines, the PCR products were inserted intact at single sites in the maize genome. The ABS loci were genetically stable and measured approximately 100 to 200 kb in size, with the largest including roughly 1300 copies of the ABS monomer (as measured by qPCR). ABS-ch3, ABS-ch4, and ABS-ch7 were located on chromosomes 3, 4 and 7, respectively. The system was also tested to confirm that it can be used to tether a protein. A Lacl-YFP fusion was transformed into maize, crossed to ABS lines and the progeny scored. Single large fluorescent spots were visible in ABS-ch3, Lacl-YFP hybrids (FIG. 1). These data establish that our tethering system is functioning.

### Example 1B

### Preparing an Engineered Centromere

A 157 bp sequence (SEQ ID NO. 6) was created that contained binding sites for five different DNA binding modules (Lacl, Gal4, LexA, TetR and CENP-B), the first four which are known to tether proteins in plants (Matzke et al. Plant Molecular Biology Reporter 21(1):9-19 (2003); Matzke et al. Plant Physiology 139(4): 1586-1596 (2005); Bohner et al. Plant J 19(1):87-95 (1999); Zuo et al. Current Opinion in Biotechnology 11(2): 146-151 (2000); Zuo et al. Methods Mol Biol 323: 329-42 (2006)). In order to multimerize the monomer, these were amplified into long Arrayed Binding Sites (called ABS) by overlapping PCR (FIG. 1). Long>200 kb PCR products composed exclusively of ABS arrays were created in-this way. Metal spheres were then coated with the PCR product and a marker plasmid, and maize calli were transformed by standard biolistic methods. In three resulting transgenic lines, the PCR products were inserted intact at single sites in the maize genome. The ABS loci were genetically stable and measured approximately 100 to 200 kb in size, with the largest including roughly 1300 copies of the ABS monomer (as measured by qPCR). ABS-ch3, ABS-cb4, and ABS-ch7 were located on chromosomes 3, 4 and 7, respectively. The system was also tested to confirm that it can be used to tether a protein. A Lacl-YFP fusion was transformed into maize, crossed to ABS lines and the progeny scored. Single large fluorescent spots were visible in ABS-ch3, Lacl-YFP hybrids (FIG. 1). These data establish that our tethering system is functioning.

### Example 2

### Tethering CENH3, CENPC, and MIS12

Three kinetochore proteins are tethered by the following methods to ABS arrays alone and in combination. A) Centromeric Histone H3. CENH3 is a histone variant and lends itself to tethering, having a long N-terminal tail that is replaceable. The construct employed is a tri-protein chimera containing a Gal4 binding module fused to an oat N-terminal tail and a maize CENH3 histone core region (Zhong et al. Plant Cell 14: 2825-2836 (2002)). Replacing the N-terminus with oat sequence allows the use of an oat CENH3 antibody. The chimeral CENH3 binds to the ABS sites, and recruits natural CENH3 to form a centromeric state that is stable after the tethered protein is removed by segregation. B) Centromere Protein C. CENPC has an important role in maize centromere assembly, and is involved in recruiting CENH3 to DNA (Dawe et al. Plant Cell 11(7): 1227-1238 (1999);Erhardt et al. J Cell Biol 183: 805-818 (2008)). A Lacl-CENPC tethering construct is employed. C) Minichromosome Instability 12. MIS12 is an important protein of the microtubule binding face in maize, regulating interactions with microtubules (Li et al. Nat Cell Biol (2009)). A LexA-MIS12 tethering construct is employed. MIS12 alone can confer chromosome segregation. D) Combinations of proteins. Each protein is fused to a different DNA binding module, so that crossing the transgenic lines results in combination of the proteins on the same ABS array. CENH3 and CENPC together can recruit the entire kinetochore complex. By combining CENH3, CENPC, and MIS12 at the same ABS sites, most if not all kinetochore functions are conferred. Without wishing to be bound by theory, these proteins are thought to bind to the ABS and in connection with kinetochore activation.

### Example 3

### Cytological and Molecular Assays of Tethered Lines

De novo kinetochore activity at ABS sites produces dicentric chromosomes (two centromeres on one chromosome), because each chromosome also has its natural centromere. Such dicentric kinetochore activity can cause chromosome breakage and visible broken chromosomes early in plant development. Since the ABS sites are heterozygous in all tests, chromosome breakage does not affect plant vigor or recovery of the chromosomes in progeny. Evidence of dicentric activity constituting proof of principle is obtained.

### Example 4

### Applications of Kinetochore Tethering

A useful artificial chromosome is synthesized by full gene synthesis. The artificial centromere within the artificial chromosome involves multiple arrayed copies of single or multiple binding sites. Such a construct need not be prepared by overlapping PCR, where every monomer is identical, but can be prepared by gene synthesis. The filler sequences between binding sites can be random or variable sequences to facilitate construction of the artificial chromosome. The transformed artificial chromosomes are activated by co-expressed tethering proteins. However, once an artificial centromere is active, it no longer needs tether constructs to remain active. The system is initially designed in maize but the approach is universal to all plants, since all components are engineered in vitro. Major uses include crop improvement and the production of medicinal proteins.

### Example 5

### Codon Optimization

The DNA binding modules chosen are derived from E. coli (LacI, LexA, TetR), yeast (Gal4) and human (CENP-B). The protein sequences of the DNA binding modules these species are preserved, but the encoding DNA sequences are changed to reflect the optimum codon usage for maize. Since prokaryotes lack a nuclear envelope, in order to assure that the proteins will be imported into plant nuclei, the nuclear localization signal to PKKKRKV are added to the fusion proteins. Epitope recognition sequences such as multimers of the HA epitope tag YPYDVPDYA can also be added.

The various methods and techniques described above provide a number of ways to carry out the application. Of course, it is to be understood that not necessarily all objectives or advantages described need be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof

In some embodiments, the numbers expressing quantities of ingredients, properties such as molecular weight, reaction conditions, and so forth, used to describe and claim certain embodiments of the application are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are reported as precisely as practicable.

In some embodiments, the terms “a” and “an” and “the” and similar references used in the context of describing a particular embodiment of the application (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

What is claimed is:

1. An engineered centromere comprising tandem repeats of a DNA sequence, comprising:
one or more binding motifs for one or more DNA binding proteins, wherein the one or more binding motifs permit binding of one or more fusion proteins comprising the DNA binding protein and a kinetochore protein to activate the engineered centromere.

2. The engineered centromere of claim 1, wherein the fusion protein further comprises a nuclear localization signal.

3. The engineered centromere of claim 2, wherein the nuclear localization signal is the nuclear localization signal to PKKRKV.

4. The engineered centromere of claim 1, wherein the fusion protein further comprises an eptitope recognition sequence.

5. The engineered centromere of claim 4, wherein the epitope recognition sequence comprises multimers of the HA epitope tag YPYDVPDYA.

6. The engineered centromere of claim 1, wherein the one or more DNA binding motifs is selected from the group consisting of TetR (SEQ ID NO. 1), CENP-B box (SEQ ID NO. 2), LacO (SEQ ID NO. 3), LexA (SEQ ID NO. 4), Gal4 (SEQ ID NO. 5), and combinations thereof

7. The engineered centromere of claim 1, wherein the DNA sequence is SEQ ID NO. 6.

8. The engineered centromere of claim 1, comprising at least 500 tandem repeats.

9. The engineered centromere of claim 1, comprising at least 1000 tandem repeats.

10. The engineered centromere of claim 1, wherein the one or more DNA binding proteins are selected from the group consisting of LacI, LexA, Gal4, TetR, CENP-B, fragments thereof and combinations thereof.

11. The engineered centromere of claim 1, wherein the one or more DNA binding proteins are selected from the group consisting of a polypeptide encoded by SEQ ID. NO. 7, amino acids 1-72 of a polypeptide encoded by SEQ ID NO. 8, amino acids 1-74 of a polypeptide encoded by SEQ ID NO. 9, amino acids 1-206 of a polypeptide encoded by SEQ ID NO. 10, amino acids 1-205 of a polypeptide encoded by SEQ ID NO. 11, and combinations thereof.

12. The engineered centromere of claim 1, wherein the one or more kinetochore proteins are selected from the group consisting of CENP-A/CENH3, CENP-C, MIS 12, CENP-O/MCM21, NDC80, CENP-S, CENP-T, NNF1, NUF2, SPC25, fragments thereof and combinations thereof.

13. A method of activating an artificial centromere, comprising:
providing the engineered centromere of claim 1; and
contacting the engineered centromere with the one or more fusion proteins comprising the one or more DNA binding proteins and the one or more kinetochore proteins, whereby the DNA binding protein portion of the one or more fusion proteins binds to engineered centromere and a kinetochore is formed.

14. A plant artificial chromosome (AC) comprising the engineered centromere of claim 1.

15. A transgenic plant comprising the artificial chromosome (AC) of claim 14.

16. The transgenic plant of claim 15, wherein the AC expresses one or more fusion proteins comprising one or more DNA binding proteins and one or more kinetochore proteins.

17. The transgenic plant of claim 15, further comprising a nucleic acid molecule capable of expressing one or more fusion proteins comprising one or more DNA binding proteins and one or more kinetochore proteins.

18. A seed carrying the artificial chromosome (AC) of claim 14.

19. A system, comprising:
an artificial centromere comprising tandem repeats of a DNA sequence comprising one or more binding motifs for one or more DNA binding proteins; and
one or more nucleic acids expressing one or more fusion proteins comprising the one or more DNA binding proteins and one or more kinetochore proteins, wherein the one or more binding motifs permit binding of the one or more fusion proteins to activate the engineered centromere to form a kinetochore.

20. The system of claim 19, wherein the fusion protein further comprises a nuclear localization signal.

21. The system of claim 20, wherein the nuclear localization signal is to PKKRKV.

22. The system of claim 19, wherein the fusion protein further comprises an eptitope recognition sequence.

23. The system of claim 22, wherein the epitope recognition sequence comprises multimers of the HA epitope tag YPYDVPDYA.

24. The system of claim 19, wherein the one or more DNA binding motifs is selected from the group consisting of TetR (SEQ ID NO. 1), CENP-B box (SEQ ID NO. 2), LacO (SEQ ID NO. 3), LexA (SEQ ID NO. 4), Gal4 (SEQ ID NO. 5), and combinations thereof.

25. The system of claim 19, wherein the DNA sequence is SEQ ID NO. 6.

26. The system of claim 19, comprising at least 500 tandem repeats.

27. The system of claim 19, comprising at least 1000 tandem repeats.

28. The system of claim 19, wherein the one or more DNA binding proteins are selected from the group consisting of LacI, LexA, Gal4, TetR, CENP-B, fragments thereof and combinations thereof.

29. The system of claim 19, wherein the one or more DNA binding proteins are selected from the group consisting of a polypeptide encoded by SEQ ID. NO. 7, amino acids 1-72 of a polypeptide encoded by SEQ ID NO. 8, amino acids 1-74 of a polypeptide encoded by SEQ ID NO. 9, amino acids 1-206 of a polypeptide encoded by SEQ ID NO. 10, amino acids 1-205 of a polypeptide encoded by SEQ ID NO. 11, and combinations thereof.

30. The system of claim 19, wherein the one or more kinetochore proteins are selected from the group consisting of CENP-A/CENH3, CENP-C, MIS12, CENP-O/MCM21, NDC80, CENP-S, CENP-T, NNF1, NUF2, SPC25, fragments thereof and combinations thereof.

31. A method of synthesizing a large molecule by adding multiple genes using the plant artificial chromosome comprising:
synthesizing an artificial chromosome;
introducing one or more recruiting constructs; and
activating the transformed artificial chromosome by co-expressing one or more fusion proteins comprising one or more DNA binding proteins and one or more kinetochore proteins.

32. The method of claim 31, wherein the artificial chromosome is synthesized by full gene synthesis.
